(19) United States
(12) Patent Application Publication
(10) Pub. No.: US 2009/0176864 A1
(43) Pub. Date: Jul. 9, 2009

(54) Methods For Improving Hepatic and Immune Function In An Animal

(75) Inventors: Steven C. Zicker, Lawrence, KS (US); Inke Paetau-Robinson, Auburn, KS (US)

Correspondence Address: COLGATE-PALMOLIVE COMPANY, 909 RIVER ROAD, PISCATAWAY, NJ, 08855, US

(73) Assignee: Hill's Pet Nutrition, Inc., Topeka, KS (US)

(21) Appl. No.: 12/353,351

(22) Filed: Jan. 14, 2009

Related U.S. Application Data
(63) Continuation-in-part of application No. 11/753,404, filed on May 24, 2007.
Application No. 11/753,404 is a continuation of application No. PCT/US05/42886, filed on Nov. 23, 2005.
(60) Provisional application No. 60/630,971, filed on Nov. 24, 2004.

(51) Int. Cl. A61K 31/385; A61P 37/00

(52) U.S. Cl. 514/440

(57) ABSTRACT

The invention encompasses compositions and methods for improving animal health and in certain embodiments to compositions and methods for improving hepatic and immune function in aged felines.

### CROSS REFERENCE TO RELATED APPLICATIONS

This application is a continuation-in-part of pending application Ser. No. 11/753,404, filed May 24, 2007, which is a continuation of PCT Application No. PCT/US05/42886, filed Nov. 23, 2005, which claims the benefit of Provisional Application Ser. No. 60/630,971 filed Nov. 24, 2004.

### FIELD OF THE INVENTION

The invention encompasses compositions and methods for improving animal health and in certain embodiments to compositions and methods for improving hepatic and immune function in aged felines.

### BACKGROUND OF THE INVENTION

The liver is a vital organ and has an important role in most every bodily function of a mammal. In one role, the litter acts as a filtration system to protect other organs from the effects of toxin buildup. Toxins absorbed from the digestive system are removed from the blood by the liver before they can affect the rest of the body. The capacity of a xenobiotic such as a drug, therapeutic agent, or chemical to produce injury to a liver is known as hepatotoxicity. The xenobiotic is a pharmacologically or toxicologically active substance not indigenously produced and therefore foreign to an organism. Many industrial compounds, drugs and other therapeutic agents are well established as injurious to a liver. As mammals age, their capacity for the filtration and clearance of xenobiotics by the liver decreases. It is well known that as mammals age, especially companion animals, they encounter health problems that require drugs and other therapeutic agents. Since liver filtration and clearance decreases in such an aged animal, administration of such drugs and therapeutic agents to improve the health of the animal may have hepatotoxic effects.

R-α-Lipoic acid (CAS number 1200-22-2, also known as thioctic acid and 1,2-dithiolane-3-pentanoic acid) naturally occurs in plant and animal tissues, where it is covalently bound to an ε-amino group of lysine residues. Lipoic acid is commercially available and is produced by companies such as BASF and Cognis. Lipoic acid is commercially available as an essentially pure R-α lipoic acid or as a racemic mixture of lipoic acid isomers. In plants, lipoic acid is most abundant in spinach and potatoes while in animal tissues, lipoic acid is most abundant in the kidney and the heart. R-α-lipoic acid was first discovered in 1937 (See Snell et al., Journal Bact. 33: 207, 1937) and was not isolated and characterized until 1951 (See Reed et al. Science 114:94-4, 1951). R-α-lipoic acid may be synthesized and such methods are well known in the art. (See U.S. Pat. No. 2,890,716 to Reed issued Apr. 18, 1961). R-α-lipoic acid has been classified as an antioxidant and has been used in high dosages as a treatment for Type II diabetes. Studies have shown that mixtures of carnitine and lipoic acid may enhance metabolism and alleviate oxidative stress. (See U.S. Pat. No. 5,916,912 to Ames et al. issued Jun. 29, 1999 and U.S. Pat. No. 6,365,622 to Cavayzo issued Apr. 2, 2002). In addition, it has been shown that a companion animal diet comprising lipoic acid among other ingredients appears to inhibit the deterioration of the mental capacity of an aged companion animal. (See U.S. Patent Application Publication Nos. 2002/0076469, 2002/0052402, 2002/0076470, 2000/115710, and 2002/0119182).

Studies have shown that mitochondrial oxidation plays a role in the metabolism of lipoic acid. Although the metabolism in humans mainly resembles that observed in mice and rats, the formation of oxidized structures related to tetranorlipoic acid found in canines appears to have no equivalent in humans. In addition, 3-ketolipoic acid, an intermediate in the mitochondrial oxidation of lipoic acid has been reported in plasma samples from rats and humans but has not been found in plasma from canines. (See Schupke, H. et al. Drug Metabolism and Disposition, 29 (6) 855-862, 2001). It appears that the metabolic pathway of α-lipoic acid is different in canines as compared to humans.

Mercapturic acids are sulfur derivatives of N-acetyl-cysteine, which is synthesized from glutathione (GSH). It is generally accepted that most compounds are metabolized to mercapturic acids first undergo conjugation with GSH catalyzed by an enzyme called glutathione S-transferase, found in the soluble or supernatant liver refractions. The mercapturic acid pathway appears to have evolved as a protective mechanism against xenobiotic induced hepatotoxicity or carcinogenicity, serving to detoxify a large number of noxious substances that are inhaled, ingested or normally produced metabolically every day. Lipoic acid not only up regulates the glutathione but also up regulates the enzyme, glutathione S-transferase that conjugates glutathione in the liver. Bromosulfophthalein (CAS number 71-67-0 also known as BSP and sulfobromophthalein) is an organic dye that, when injected into the circulation, is removed by the liver at a rate that reflects the liver's ability to extract and metabolize a number of organic compounds. See S. M. Rosenthal, E. C. White, J. Pharmacol. 24, 265 (1924) W. Hacki et al., J. Lab. Clin. Med. 88, 1019 (1976). BSP is cleared from the liver in three steps. First, BSP is transferred from albumin through the plasma to the liver. This step is dependent on plasma protein concentration and other ligands that bind to plasma proteins. Secondly, BSP is complexed in the liver by a ligand and z protein. Finally, BSP is conjugated by glutathione via glutathione S-transferase enzyme and eliminated into the bile duct and this is the rate-limiting step. Thus BSP is an example of a xenobiotic that, when measured in the blood after injection, provides information on the functional capabilities of the liver.

### SUMMARY OF THE INVENTION

The invention generally encompasses compositions comprising an effective amount of lipoic acid or a salt thereof, wherein said effective amount is effective in improving hepatic or immune function in an animal.

In another embodiment, the invention encompasses methods for improving hepatic function in an animal in need thereof by feeding lipoic acid or a salt thereof to the animal, generally in a diet including lipoic acid or a salt thereof in an amount effective to improve hepatic function.

In another embodiment, the invention encompasses methods for improving immune function in an animal in need thereof by feeding lipoic acid or a salt thereof to the animal, generally in a diet comprising lipoic acid or a salt thereof in an amount effective to improve immune function.

In various embodiments, the invention is a new approach for improving the health of aging animals, for example felines, based upon the use of lipoic acid or a salt thereof as part of a diet that is fed to the animals.

Further areas of applicability of the present invention will become apparent from the detailed description provided hereinafter. It should be understood that the detailed description and specific examples, while indicating the illustrative embodiments of the invention, are not intended to limit the scope of the invention.

### BRIEF DESCRIPTION OF THE DRAWINGS

FIG. 1 is a graphical representation illustrating that the inclusion of lipoic acid into foods at 65 ppm and 650 ppm for 6 weeks had no adverse effects on bodyweight for cats.

FIG. 2 illustrates the effect of inclusion of 65 ppm and 650 ppm in diets and time on Concanavalin A stimulation. There was no significant difference between the groups at the beginning of the study or at the end. However, the cats on the 65 ppm lipoic acid inclusion had a significant increase in Concanavalin A-activated lymphocyte proliferation compared to baseline. Cats on the 650 ppm also displayed an increase.

FIG. 3 illustrates that phytohaemagglutinin (PHA) stimulation showed no significant difference between groups at beginning or end. The illustrative group administered 65 ppm lipoic acid had a significant increase in lymphocyte proliferation between baseline and 6 weeks of intervention.

FIG. 4 illustrates Pokeweed Mitogen stimulation was different between groups (ANOVA P<0.05) at the beginning but not the end of the study. The group with the lowest starting mean was the 65 ppm group. Subsequently, lymphocyte proliferation in the group administered 65 ppm lipoic acid was significantly increased compared to baseline after 6 weeks time.

FIGS. 5a and 5b illustrate natural killer cell activity. Based on the illustrative studies, no significant changes were detected from baseline to end of study for the 10:1 stimulation rate; however, all p values were less than 0.1. All changes between baseline and end of study were significant for all groups at the 50:1 stimulation rate.

FIG. 6 illustrates graphs for representative Comet assays. Two illustrative assays were performed: (1) inherent DNA damage and (2) hydrogen peroxide challenged damage. Analysis of the data showed that all head DNA for all diets increased significantly (P<0.05) over the duration of the study for both. In addition, all tail DNA tail length and Olive tail moments decreased during the duration of the study for both control and hydrogen peroxide challenged comet tests. However, there were no significant differences between groups at either the beginning or end for any of the comet measures. In addition, ANOVA analysis of the change over time (difference pre-post) showed no significant difference via t-test for each comet variable under control and hydrogen peroxide challenge conditions.

The Figures are intended to exemplify the general characteristics of the invention for the purposes of the description of such embodiments herein. The Figure may not precisely reflect the characteristics of any given embodiment and is not necessarily intended to define or limit specific embodiments within the scope of this invention.

### DETAILED DESCRIPTION OF THE INVENTION

### Definitions

The term “animal” means any animal susceptible to or suffering from impaired liver function and in need of improved liver clearance of xenobiotic substances or an animal that could benefit from improved liver clearance of xenobiotic substances. An animal is “susceptible to” a disease or condition if the animal exhibits symptoms that indicate that the animal is likely to develop the condition or disease. An animal is “suffering from” a disease or condition if the animal exhibits symptoms that are indicative that the animal has developed the condition or disease.

As use herein, the terms “lipoic acid or a salt thereof” includes, but is not limited to, for example, alpha-lipoic acid, a racemic mixture of lipoic acids, a lipoate salt, ester, amide or derivative thereof, for example as described in U.S. Pat. No. 5,621,117. In various embodiments, the lipoic acid can be administered in a composition comprising a wet or dry food composition, which may be in the form of a moist food, dry food, supplement or treat. The lipoic acid may be incorporated therein or on the surface of any food composition, such as, by spraying or precipitation thereon or may be added to the diet by way of snack, supplement, treat or in the liquid portion of the diet such as water or another fluid. The lipoic acid may be administered as a powder, solid or as a liquid including a gel. An important aspect is that the animal be provided an effective amount of the lipoic acid to provide a positive effect. Typically, the source of lipoic acid or a salt thereof is present in the composition in an amount of up to an amount which remains non-toxic to the animal.

The phrase “salt thereof,” as used herein includes but is not limited to salts of lipoic acid used in the pet food compositions. Lipoic acid is acidic in nature and therefore is capable of forming base salts with various cations. Examples of such salts include alkali metal or alkaline earth metal salts and, particularly, calcium, magnesium, sodium lithium, zinc, potassium, and iron salts.

The term “older animal” means any animal susceptible to or suffering from impaired liver function and in need of improved liver clearance of xenobiotic substances or an animal that could benefit from improved liver clearance of xenobiotic substances because of age.

### GENERAL DESCRIPTION

The invention generally encompasses compositions comprising an effective amount of lipoic acid or a salt thereof, wherein said effective amount is effective in improving hepatic or immune function in an animal.

In certain embodiments, the effective amount is effective in improving hepatic function in an animal.

In other embodiments, the effective amount is effective in improving immune function in an animal.

In another embodiment, the effective amount is from 25 ppm to 2600 ppm of lipoic acid or a salt thereof.

In another embodiment, the effective amount is from 50 ppm to 1200 ppm of lipoic acid or a salt thereof.

In another embodiment, the effective amount is from 65 ppm to 650 ppm of lipoic acid or a salt thereof.

In another embodiment, the animal is a companion animal.

In another embodiment, the companion animal is a feline.

In another embodiment, the composition is a food composition.

In another embodiment, the food composition is suitable for a companion animal.

In another embodiment, the food composition is extruded.

In another embodiment, the food composition is calmed.

In another embodiment, the invention encompasses methods for improving hepatic or immune function in animal comprising feeding an effective amount of lipoic acid or a salt thereof to the animal, wherein said effective amount is effective in improving hepatic or immune function.

In certain embodiments, the methods are effective in improving hepatic function in an animal.

In other embodiments, the methods are effective in improving immune function in an animal.

In another embodiment, the effective amount is from 25 ppm to 2600 ppm of lipoic acid or a salt thereof.

In another embodiment, the effective amount is from 50 ppm to 1200 ppm of lipoic acid or a salt thereof.

In another embodiment, the effective amount is from 65 ppm to 650 ppm of lipoic acid or a salt thereof.

In another embodiment, the animal is a companion animal.

In another embodiment, the companion animal is a feline.

In another embodiment, the lipoic acid is part of the animal's daily diet.

In another embodiment, the daily diet comprises lipoic acid in an amount of greater than 50 ppm on a dry weight basis.

In another embodiment, the lipoic acid is fed to the animal in a food composition suitable for consumption by the animal.

In another embodiment, the animal is an older animal.

Another embodiment encompasses a composition suitable for improving immune function in an animal comprising an amount of nutrients and greater than 50 ppm of lipoic acid.

In certain embodiments, the composition is a food composition.

In other embodiments, the food composition is suitable for a companion animal.

In another embodiment, the food composition is suitable for a feline.

In another embodiment, the composition is extruded or canned.

### COMPOSITIONS OF THE INVENTION

One embodiment of the invention encompasses compositions for companion animals including an effective amount of lipoic acid or a salt thereof to improve hepatic or immune function in an animal.

The quantity of alpha-lipoic acid in the compositions can vary from at least about 25 ppm, about 50 ppm, about 100 ppm, about 200 ppm, about 300 ppm, about 500 ppm, about 700 ppm, about 900 ppm, about 1100 ppm, about 1200 ppm, about 1400 ppm, about 1600 ppm, about 1800 ppm, about 2000 ppm, about 2200 ppm, about 2400 ppm, or about 2600 ppm.

In various embodiments, the range of lipoic acid that can be administered to cats is 25 ppm to 2600 ppm. In certain illustrative embodiments, quantities can vary 65 ppm to an amount which remains nontoxic to the pet. In other embodiments, a range is 50 ppm to 1200 ppm. In other embodiments, a range is 65 ppm to 650 ppm.

In various embodiments, a food composition comprising lipoic acid provides a substantially nutritionally complete diet for the intended recipient animal. A “nutritionally complete diet” is a diet that includes sufficient nutrients for maintenance of normal health of a healthy animal on the diet.

The lipoic acid or salt thereof is present at a concentration that is not deleterious to the intended animal's health. Thus, for example, the lipoic acid or salt thereof is present at a concentration that does not cause undesirable or toxic effects.

The composition can be a liquid or a solid food. When the composition is a liquid, the lipoic acid or salt thereof can be admixed with other components. Where the composition is solid, the lipoic acid may be coated on the composition, incorporated into the composition, or both.

In various embodiments, the lipoic acid or salt thereof may be added to the animal's food. In certain embodiments, the lipoic acid or salt thereof may be added to the animal's food by a compounder or manufacturer at a site or by an animal's caregiver prior to feeding the animal. In other embodiments, the lipoic acid or salt thereof may be added during the processing of an animal's food, such as during and/or after mixing of other components of the composition that is then packaged and made available to consumers. Such processing may include extrusion, canning, baking, and the like or any other method or process of producing pet foods that is known in the art. In other embodiments, the lipoic acid or salt thereof may be contributed by a natural source like an animal or plant component, or the lipoic acid or salt thereof may be contributed by a synthetically derived source, or the lipoic acid or salt thereof may be contributed by a mixture of natural and synthetic sources.

The compositions in addition to lipoic acid or a salt thereof include at least one component suitable for consumption by a companion animal including, but not limited to, fats, carbohydrates, proteins, fibers, nutritional balancing agents such as vitamins, minerals, and trace elements, and mixtures thereof. One of ordinary skill in the art can select the amount and type of food ingredients for a typical food based upon the dietary requirements of the animal, for example, the animal's species, age, size, weight, health, and function.

The food ingredient part of the food composition can include up to about 100% of any particular food ingredient or can include a mixture of food ingredients in various proportions. In certain embodiments, the food composition includes a combination of food ingredients in amounts of 0 wt. % to 50 wt. % fat, 0 wt. % to 75 wt. % carbohydrate. 0 wt. % to 95 wt. % protein, 0 wt. % to 40 wt. % dietary fiber, and 0 wt. % to 15 wt. % of one or more nutritional balancing agents.

In certain embodiments, the fat and carbohydrate food ingredient is obtained from a variety of sources such as animal fat, fish oil, vegetable oil, meat, meat by-products, grains, other animal or plant sources, and mixtures thereof. Grains include wheat, corn, barley, and rice.

In certain embodiments, the protein food ingredient is obtained from a variety sources such as plants, animals, or both. Animal protein includes meat, meat by-products, dairy, and eggs. Meats include the flesh from poultry, fish, and animals such as cattle, swine, sheep, goats, and the like, meat by-products include lungs, kidneys, brain, livers, stomachs, and intestines. The protein food ingredient may also be free amino acids and/or peptides. Preferably, the protein food ingredient includes meat, a meat by-product, dairy products, or eggs.

In certain embodiments, the fiber food ingredient is obtained from a variety of sources such as vegetable fiber sources, for example, cellulose, beet pulp, peanut hulls, and soy fiber.

In certain embodiments, the nutritional balancing agents are obtained from a variety of sources known to skilled artisans, for example, vitamin and mineral supplements and food ingredients. Vitamins and minerals can be included in amounts required to avoid deficiency and maintain health. These amounts are readily available in the art. The National Research Council (NRC) provides recommended amounts of such nutrients for farm animals. See, e. g., Nutrient Requirements of Swine (10th Rev. Ed., Nat'l Academy Press, Wash. D.C., 1998), Nutrient Requirements Of Poultry (9th Rev. Ed., Nat'l Academy Press, Wash. D.C., 1994). Nutrient Requirements of Horses (5th Rev. Ed., Nat'l Academy Press, Wash. D.C., 1989). The American Feed Control Officials (AAFCO) provides recommended amounts of such nutrients for dogs and cats. See American Feed Control Officials, Inc., Official publication, pp. 129-137 (2004). Vitamins generally useful as food additives include vitamin A, vitamin B1, vitamin B2, vitamin B6, vitamin B12, vitamin D, biotin, vitamin K, folic acid, inositol, niacin, and pantothenic acid. Minerals and trace elements useful as food additives include calcium, phosphorus, sodium, potassium, magnesium, copper, zinc, chloride, iron, selenium, iodine, and iron.

### PREPARATION OF THE COMPOSITIONS OF THE INVENTION

The compositions of the invention may be prepared in a canned or wet form using conventional food preparation processes known to skilled artisans. Typically, ground animal proteinaceous tissues are mixed with the other ingredients such as fish oils, cereal grains, balancing ingredients, special purpose additives (e.g., vitamin and mineral mixtures, inorganic salts, cellulose and beet pulp, bulking agents, and the like) and water in amounts sufficient for processing. These ingredients are mixed in a vessel suitable for heating while blending the components. Heating of the mixture is effected using any suitable manner, for example, direct steam injection or using a vessel fitted with a heat exchanger. Following the addition of the last ingredient, the mixture is heated to a temperature of about 50° F. to about 212° F. Temperatures outside this range are acceptable but may be commercially impractical without use of other processing aids. When heated to the appropriate temperature, the material will typically be in the form of a thick liquid. The thick liquid is filled into cans. A lid is applied, and the container is hermetically sealed. The sealed can is then placed into conventional equipment designed to sterilize the contents. Sterilization is usually accomplished by heating to temperatures of greater than about 230° F. for an appropriate time depending on the temperature used, the composition, and similar factors. The compositions of the present invention can be added to the food compositions before, during, or after preparation.

Food compositions may be prepared in a dry form using conventional processes known to skilled artisans. Typically, dry ingredients such as animal protein, plant protein, grains, and the like are ground and mixed together. Moist or liquid ingredients, including fiats, oils, animal protein, water, and the like are then added to and mixed with the dry mix. The mixture is then processed into kibbles or similar dry pieces. Kibble is often formed using an extrusion process in which the mixture of dry and wet ingredients is subjected to mechanical work at a high pressure and temperature and forced through small openings and cut off into kibble by a rotating knife. The wet kibble is then dried and optionally coated with one or more topical coatings such as flavors, fats, oils, powders, and the like. Kibble also can be made from the dough using a baking process, rather than extrusion, wherein the dough is placed into a mold before dry-heat processing. The food compositions can be in the form of a treat using an extrusion or baking process similar to those described above for dry food or a toy such as those disclosed in U.S. Pat. Nos. 5,339,771 and 5,419,283. The compositions of the present invention can be added to the food compositions before, during, or after preparation.

### METHODS OF THE INVENTION

The invention also encompasses methods for improving hepatic function in animals. The methods include feeding an amount of lipoic acid or a salt thereof effective to improve hepatic function to an animal in need thereof. Generally, the lipoic acid is feed to the animal in amounts of 25 ppm to 2600 ppm. In certain illustrative embodiments, quantities can vary from 25 ppm to 2600 ppm or to an amount which remains nontoxic to the pet. In other embodiments, a range is 50 ppm to 1200 ppm. In other embodiments, a range is 65 ppm to 650 ppm.

The invention also encompasses methods for improving immune function in animals. The methods include feeding an amount of lipoic acid or a salt thereof in an amount effective to improve immune function to the animal in need thereof. Generally, the lipoic acid is feed to the animal in amounts of 25 ppm to 2600 ppm. In certain illustrative embodiments, quantities can vary from 25 ppm to 2600 ppm or to an amount which remains nontoxic to the pet. In other embodiments, a range is 50 ppm to 1200 ppm. In other embodiments, a range is 65 ppm to 650 ppm.

The methods of the invention include feeding an animal, for example, a companion animal such as a feline, a composition or diet containing lipoic acid or a salt thereof to improve hepatic function or immune function, particularly when these functions may be impaired by age, and to improve the overall health of the animal. The amount of lipoic acid given to the animal is a non-toxic amount. The lipoic acid may be either provided to the animal as a supplement or contained in a composition, including a diet, fed to the animal. Such a supplement may be in the form of a pill or capsule, a treat or a biscuit, or any other edible form. By “diet,” it is meant the food or drink regularly consumed by the animal. A diet may include supplements consumed by the animal. A diet is considered to have essentially enough nutrients to be life sustaining for the animal. A companion animal diet can be any suitable pet food formula, which also provides adequate nutrition for the animal. For example, a typical feline diet for use in the present invention may contain from 8 to 50% fat, 16 to 50% by weight protein and 3 to 15% total dietary fiber. In another example, a typical feline diet may contain from 8 to 50% by weight fat and from 30 to 60% by weight protein. However, no specific ratios or percentages of these or other nutrients are required. A nutrient is any food constituent that helps support life. Nutrients important to an animal's health are known to skilled artisans, for example, proteins, carbohydrates, fats, fibers, vitamins, and minerals. Water is also vital to an animal's health.

Various embodiments of the invention include a method for improving hepatic function or immune function in an animal, particularly a companion animal. In such embodiments, the method comprises feeding to the animal a composition, for example a diet, comprising lipoic acid or a salt thereof in an amount of at least 25 ppm on a dry matter basis. In still other embodiments, the method comprises feeding to the animal a diet comprising lipoic acid in an amount from 65 ppm to 650 ppm on a dry matter basis. As used herein, lipoic acid is in a racemic mixture, but other embodiments may include lipoic acid which is essentially pure R-α lipoic acid or as a lipoate derivative, mixtures of isomers, salts, esters, amides or combinations thereof (For example see U.S. Pat. No. 5,621,177 to Bethge et al. issued Apr. 15, 1997). In various embodiments, the range of lipoic acid that can be administered cats is 25 ppm to 2600 ppm. In certain illustrative embodiments, quantities can vary from 65 ppm to 2600 ppm or to an amount which remains nontoxic to the pet. In other embodiments, a range is 50 ppm to 1200 ppm. In other embodiments, a range is 65 ppm to 650 ppm.

In various embodiments, a composition or diet comprising at least 25 ppm to 2600 ppm of lipoic acid or a salt thereof. In some embodiments, the lipoic acid or salt thereof is added to the companion animal's food. In such embodiments, the lipoic acid or salt thereof may be added during the processing of the companion animal food that is then packaged and made available to consumers. Such processes may include extrusion, canning, baking and the like or any other method or process of producing pet foods that is known in the art. In such processes, the lipoic acid may be contributed by a natural source like an animal or plant component, such as kidney or spinach or the lipoic acid may be contributed by a synthetically derived source, or the lipoic acid may be contributed by a mixture of natural and synthetic sources. In other embodiments, lipoic acid may be in a capsule form to be fed to the companion animal. In still other embodiments, the lipoic acid or salt thereof may be in a powder or in a crystalline, which may be added to the animal's food or fed directly to the animal. In various embodiments, the companion animal diet comprises lipoic acid or salt thereof and other needed nutritional components. In various embodiments, the companion animal is a dog and in other embodiments, the companion animal is a cat.

In a further aspect, the present invention provides for a use of lipoic acid or salt thereof to prepare a medicament. In another, the invention provides for the use of lipoic acid to prepare a medicament for maintaining and/or improving animal health, e.g., improving hepatic function or immune function in an animal by feeding an amount of lipoic acid or a salt thereof to the animal. Generally, medicaments are prepared by admixing a compound or composition with excipients, buffers, blinders, plasticizers, colorants, diluents, compressing agents, lubricants, flavorants, moistening agents, and other ingredients known to skilled artisans to be useful for producing medicaments and formulating medicaments that are suitable for administration to an animal.

In a further aspect, the invention provides kits suitable for feeding lipoic acid or salt thereof to an animal. The kits comprise in separate containers in a single package or in separate containers in a virtual package, as appropriate, lipoic acid and at least one of (1) one or more ingredients suitable for consumption by an animal, (2) instructions for how to combine the lipoic acid and other kit components to improve liver clearance of xenobiotic substances, particularly to produce a composition useful for improving liver clearance of xenobiotic substances, and (3) instructions for how to use the lipoic acid and other components of the present invention, particularly for the benefit of the animal. When the kit comprises a virtual package, the kit is limited to instructions in a virtual environment in combination with one or more physical kit components. The kit contains the lipoic acid and other components in amounts sufficient to improve liver clearance of xenobiotic substances. Typically, the lipoic acid and the other suitable kit components are admixed just prior to consumption by an animal. In one embodiment, the kit contains a packet containing lipoic acid and a container of food for consumption by an animal. The kit may contain additional items such as a device for mixing the lipoic acid and ingredients or a device for containing the admixture, e.g., a food bowl. In another embodiment, the lipoic acid is mixed with additional nutritional supplements such as vitamins and minerals that promote good health in an animal.

This invention is not limited to the particular methodology, protocols, and reagents described herein because they may vary. Further, the terminology used herein is for the purpose of describing particular embodiments only and is not intended to limit the scope of the present invention. As used herein and in the appended claims, the singular forms “a,” “an,” and “the” include plural reference unless the context clearly dictates otherwise. The terms “comprise,” “comprises”, and “comprising” are to be interpreted inclusively rather than exclusively.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the preferred methods, devices, and materials are described herein.

All patents, patent applications, and publications mentioned herein are incorporated herein by reference to the extent allowed by law for the purpose of describing and disclosing the compositions, compounds, methods, and similar information reported therein that might be used with the present invention. However, nothing herein is to be construed as an admission that the invention is not entitled to antedate such disclosure by virtue of prior invention.

### EXAMPLES

This invention can be further illustrated by the following examples of preferred embodiments thereof, although it will be understood that these examples are included merely for purposes of illustration and are not intended to limit the scope of the invention unless otherwise specifically indicated.

### Example 1

The study involved three groups of cats: Group 1) cats on a dry control food, Group 2) cats on a dry food fortified with approximately 65 ppm of lipoic acid, and Group 3) cats on a dry food fortified with approximately 650 ppm of lipoic acid on a dry matter basis. All cats were fled control food for a two week baseline period at the end of which time immune function assays were performed. One group of cats was then switched to the 65 ppm test food, one group to the 650 ppm test food and one group remained on control and all were fed for another 6 weeks at which time the baseline tests were performed again.

The administration of lipoic acid to old cats improved lymphocyte proliferation activity, which may improve immune function. Most notably the effect of lipoic acid on improved lymphocyte proliferation in healthy cats at a single level of inclusion (e.g., 65 ppm).

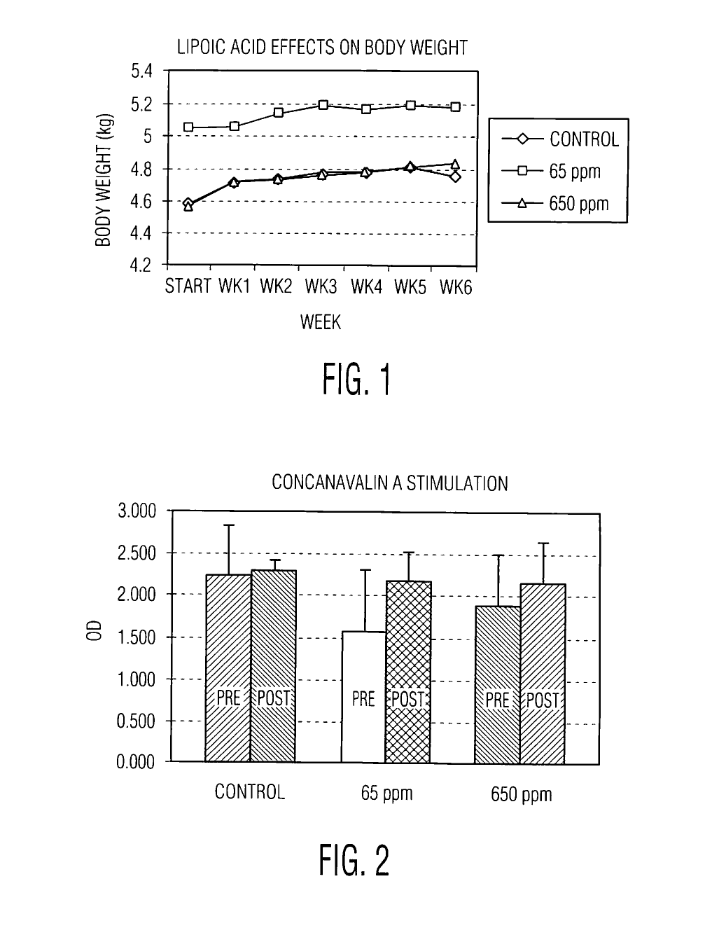

As illustrated in FIG. 1, the inclusion of lipoic acid into feline pet foods at 65 ppm and 650 ppm for 6 weeks had no adverse effects on bodyweight for cats.

FIG. 2 illustrates the effect of inclusion of 65 ppm and 650 ppm in diets and time on Concanavalin A stimulation. There was no significant difference between the groups at the beginning of the study or at the end. However, the cats on the 65 ppm lipoic acid inclusion had a significant increase in Concanavalin A-activated lymphocyte proliferation compared to baseline. Cats on the 650 ppm also displayed an increase. Accordingly based on the Concanavalin A-activated lymphocyte proliferation, the addition of 65 ppm or 650 ppm lipoic acid to a cat food Composition could increase the immune response of cats.

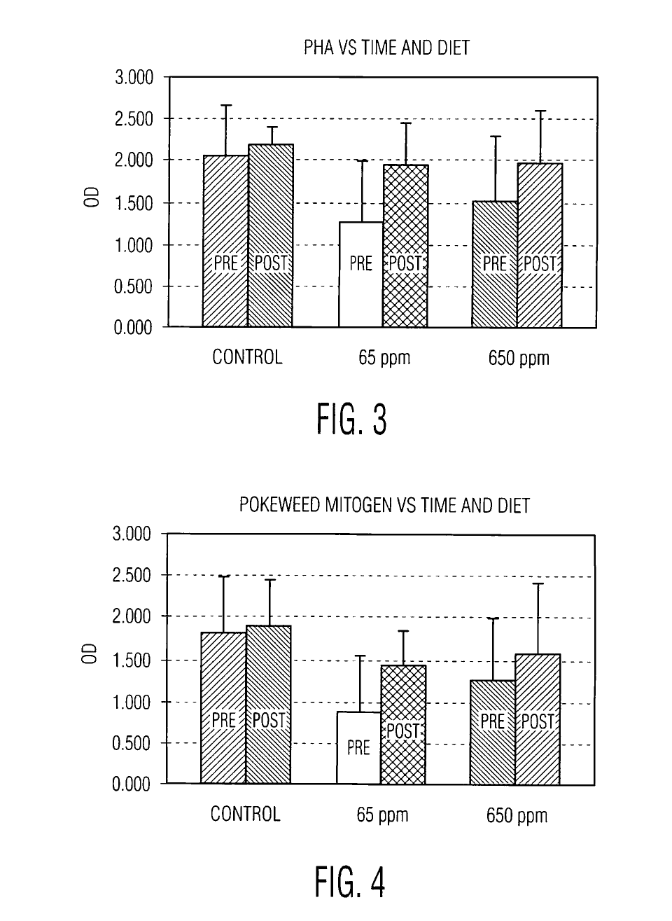

FIG. 3 illustrates that phytohaemagglutinin (PHA) stimulation showed no significant difference between groups at beginning or end. The illustrative group administered 65 ppm lipoic acid had a significant increase in lymphocyte proliferation between baseline and 6 weeks of intervention. Accordingly, based on the PHA-activated lymphocyte proliferation, the addition of 65 ppm or 650 ppm lipoic acid to a cat food composition could increase the immune response of cats

FIG. 4 illustrates Pokeweed Mitogen stimulation was different between groups (ANOVA P<0.05) at the beginning but not the end of the study. The group with the lowest starting mean was the 65 ppm group. Subsequently, lymphocyte proliferation in the group administered 65 ppm lipoic acid was significantly increased compared to baseline after 6 weeks time. Accordingly, based on the Pokeweed mitogen-activated lymphocyte proliferation, the addition of 65 ppm or 650 ppm lipoic acid to a cat food composition could increase the immune response of cats

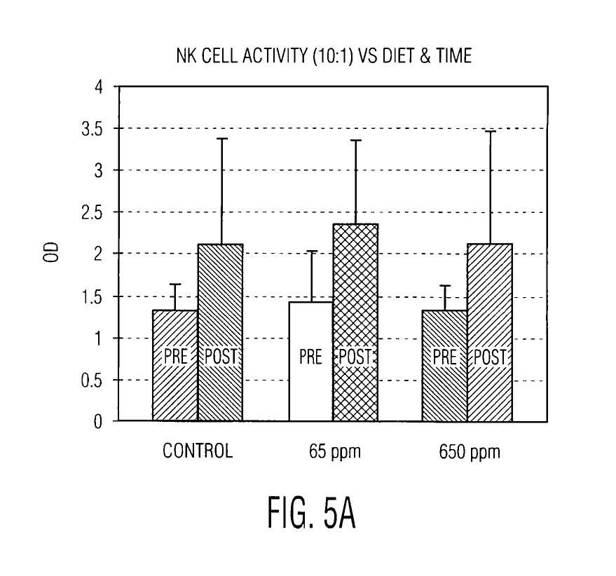

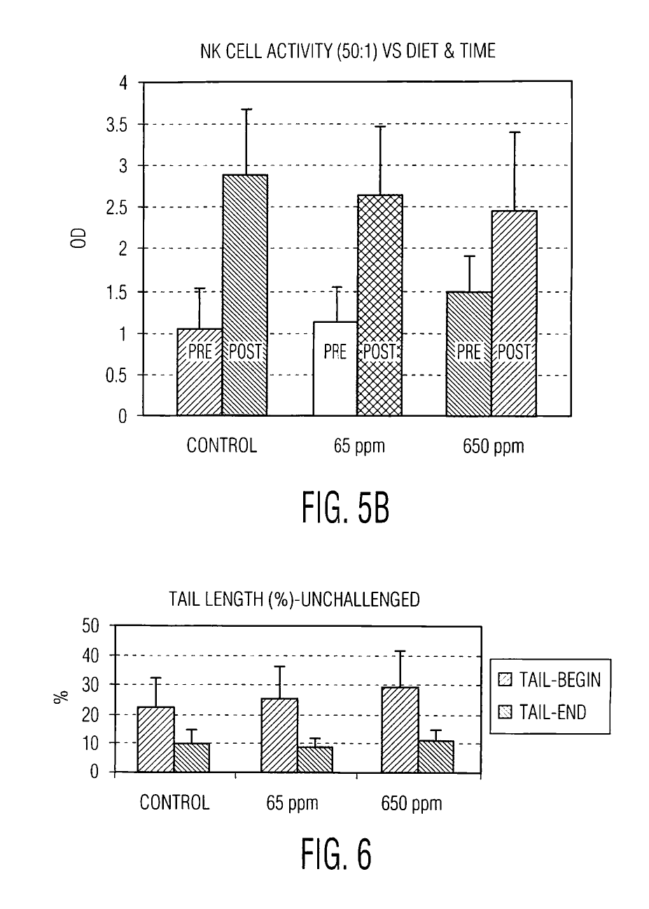

FIGS. 5a and 5b illustrate natural killer cell activity. Based on the illustrative studies, no significant changes were detected from baseline to end of study for the 10:1 stimulation rate; however, all p values were less than 0.1. All changes between baseline and end of study were significant for all groups at the 50:1 stimulation rate.

FIG. 6 illustrates graphs for representative Comet assays. Two illustrative assays were performed: (1) inherent DNA damage and (2) hydrogen peroxide challenged damage. Analysis of the data showed that all head DNA for all diets increased significantly (P<0.05) over the duration of the study for both. In addition, all tail DNA tail length and Olive tail moments decreased during the duration of the study for both control and hydrogen peroxide challenged comet tests. However, there were no significant differences between groups at either the beginning or end for any of the comet measures. In addition, ANOVA analysis of the change over time (difference pre-post) showed no significant difference via t-test for each comet variable under control and hydrogen peroxide challenge conditions.

In the specification, there have been disclosed typical preferred embodiments of the invention and, although specific terms are employed, they are used in a generic and descriptive sense only and not for purposes of limitation, the scope of the invention being set forth in the following claims. Obviously many modifications and variations of the present invention are possible in light of the above teachings. It is therefore to be understood that within the scope of the appended claims the invention may be practiced otherwise than as specifically described.

What is claimed is:

1. A composition comprising an effective amount of lipoic acid or a salt thereof to improve hepatic or immune function in an animal, wherein said effective amount of lipoic acid to improve hepatic or immune function in an animal is at least about 25 ppm.

2. The composition of claim 1, wherein the effective amount is from 25 ppm to 2600 ppm of lipoic acid or a salt thereof.

3. The composition of claim 1, wherein the effective amount is from 50 ppm to 1200 ppm of lipoic acid or a salt thereof.

4. The composition of claim 1, wherein the effective amount is from 65 ppm to 650 ppm of lipoic acid or a salt thereof.

5. The composition of claim 1, wherein the animal is a companion animal.

6. The composition of claim 5, wherein the companion animal is a feline.

7. The composition of claim 1, wherein the composition is a food composition.

8. The composition of claim 7, wherein the food composition is a dry food composition.

9. The composition of claim 7, wherein the food composition is extruded.

10. The composition of claim 7, wherein the food composition is canned.

11. A method for improving hepatic function or immune function in an animal in need thereof comprising feeding an effective amount of lipoic acid or a salt thereof to the animal, wherein said effective amount is effective in improving hepatic or immune function.

12. The method of claim 11, wherein the effective amount is from 25 ppm to 2600 ppm of lipoic acid or a salt thereof.

13. The method of claim 11, wherein the effective amount is from 50 ppm to 1200 ppm of lipoic acid or a salt thereof.

14. The method of claim 11, wherein the effective amount is from 65 ppm to 650 ppm of lipoic acid or a salt thereof.

15. The method of claim 11, wherein the animal is a companion animal.

16. The method of claim 15, wherein the companion animal is a feline.

17. The method of claim 1, wherein the lipoic acid or salt thereof is part of the animal's daily diet.

18. The method of claim 17, wherein the daily diet comprises lipoic acid or salt thereof in an amount of greater than 25 ppm on a dry weight basis.

19. The method of claim 11, wherein the lipoic acid or salt thereof is in a dry food composition.

20. The method of claim 11, wherein the animal is an older animal.

21. A composition suitable for improving immune function in an animal comprising: an amount of nutrients; and greater than 25 ppm of lipoic acid or a salt thereof.

22. The composition of claim 21 wherein the composition is a food composition.

23. The composition of claim 21, wherein the food composition is suitable for a companion animal.

24. The composition of claim 21, wherein the food composition is suitable for a feline.

25. The composition of claim 21, wherein the composition is extruded or canned.
